(19) United States
(12) Patent Application Publication
(10) Pub. No.: US 2011/0263047 A1
(43) Pub. Date: Oct. 27, 2011

(54) PEPTIDE TAG AND USES THEREOF

(76) Inventor: Axel Knebel, Dundee (GB)

(21) Appl. No.: 13/141,899

(22) PCT Filed: Dec. 23, 2009
(86) PCT No.: PCT/GB2009/002950

§ 371 Date: Jun. 23, 2011

(30) Foreign Application Priority Data
Dec. 24, 2008 (GB) 0823550.9

(51) Int. Cl. G01N 33/566; C12P 21/00; C12N 5/10; C12N 15/63; C12N 1/00

(52) U.S. Cl. 436/501; 435/320.1; 435/243; 435/325; 435/419; 435/69.7

(57) ABSTRACT

The present invention provides a novel peptide tag, uses of the same and methods and systems for the purification of proteins. In particular, the invention provides the use of a beta-lactam (β-lactam) binding protein or fragment, analogue, homologue, variant or derivative thereof, as a tag or label.

### FIELD OF THE INVENTION

The present invention provides a novel peptide tag, uses of the same and methods and systems for the purification of proteins.

### BACKGROUND OF THE INVENTION

In order to purify ectopically expressed proteins from cells, they are normally tagged, so that affinity purification can be used, which is both faster and more efficient than other purification methods. It is normally not especially difficult to obtain pure tagged proteins from bacterial expression systems, but it is much more challenging to quickly obtain proteins with high purity and yield from eukaryotic cells, particularly from transfected mammalian cells. This is because the eukaryotic proteome is much more complex than the prokaryotic, so that many more contaminant species exist. However, for some applications the expression in and purification from bacteria is not an option. For example, for the identification and investigation of physiological binding partners and post translational modifications relevant mammalian cells must be used. Also expression in and purification from eukaryotic cells may provide a means of obtaining fully active enzymes, which otherwise would have to be activated in vitro and subsequently repurified. For example a protein kinase may be expressed as a tagged fusion protein and activated by the treatment of the host cells with an agonist, e.g. a growth factor or cytokine and subsequently purified to homogeneity. This cannot be done in bacterial or insect cell expression systems, because the receptors and necessary signal transduction pathways do not exist there.

A number of tags are commonly used for the affinity purification of proteins. Cellular glutathione S-transferase (GST), was purified for many years using its reversible binding properties to glutathione sepharose (Clark et al., 1977). In 1988 Smith at Amrad, Australia exploited this interaction for the development of a tag for the purification of any protein by means of GST-fusion (Smith and Johnson, 1988; see also EP0293249 and WO8809372), which can be bound to GSH-sepharose and eluted with reduced glutathione (GSH). Smith also introduced the idea of a protease cleavage site, so that the tag may be removed if required. The method works very well for the expression and purification of proteins in E. coli, but is much less useful in eukaryotic systems, because eukaryotic cells contain large quantities of cellular glutathione S transferase, and Carbonyl reductase, both of which compete with the fusion protein for the immobilised GSH, thereby strongly reducing and contaminating yields. Furthermore, because the cells do also contain reduced GSH, the interaction of the fusion proteins to affinity media is partially inhibited. Another commonly used tag, the Histidine₆-tag, consisting of a short aminoacid repeat of 6 or more histidine residues is bound to Ni⁺⁺-agarose or other metal chelate columns and eluted with fairly high concentrations of imidazole. This method (Hochuli et al., 1987; Bush et al., 1991; Crowe et al., 1994; U.S. Pat. No. 5,284,933) is at best an enrichment method, because a large amount of cellular proteins bind to Ni²⁺-agarose and thereby contaminate the preparations. Sometimes cellular contaminants bind via Ni²⁺-agarose via their natural histidine repeat sequences but also because via negatively charges side groups, because Ni²⁺-agarose is an anion exchanger. For most applications the eluent—imidazole—must be removed, which introduces a second purification step. Sometimes, in order to achieve sufficient purity, the GST-tag and the His-tag are combined and used consecutively. For increased purity, protease cleavage is useful, as it excludes contamination with unspecific binders.

More specific purification methods utilize small peptide tags, referred to as epitope tags, which are recognized by specific antibodies. The sequence EQKLISEEDL is a c-terminal fragment of the Myc protein and hence referred to as Myc-tag, which is recognised specifically by the monoclonal antibody 9E10 (Ellison and Hochstrasser, 1991). Another commonly used peptide YPYDVPDYASL is a fragment of the influenza virus hemagglutinin protein, recognised by the monoclonal antibody 12CA5 and referred to as HA-tag (Chen et al., 1993). There are several other epitope tags sharing the principle of using a specific aminoacid sequence and a monoclonal antibody for recognition. These tags are good for immuno detection by western blotting, but not for protein purification, because there are obvious problems. The proteins must be eluted either by denaturing the immobilised antibodies, thereby potentially destroying the activity and structure of the fusion protein, or by eluting with antibodies. The latter is not only expensive but of course contaminates the fusion protein with the antibodies. This approach is therefore not suitable for scale up and rarely used for protein purification.

In order to obtain very pure native fusion proteins, tandem affinity purification (TAP) methods were introduced. These methods share a principle by using two or three different tags e.g. GST, His₆, Ca⁺⁺ binding domains, streptavidin, protein G binding domains, HA or Myc, sometimes separated by a protease cleavage site and the purification of the proteins over several subsequent columns (e.g. Rigaut et al., 1999; Honey et al., 2001; Puig et al., 2001). These methods have shown great usefulness in obtaining very pure protein, but are of course more complex than a one step procedure and also more expensive. Importantly, they are difficult to scale up and the yield is diminished by the need of at least two chromatographic steps. Long incubation during the complex purification may be harmful for protein integrity and activity.

### SUMMARY OF THE INVENTION

To establish a straight forward, one step, cost efficient and scalable procedure, which produces pure tagged protein expressed in eukaryotic and/or prokaryotic cells, a method is required where a cheap small molecule ligand binds exclusively and reversibly to a tag, but not to other cellular proteins. Such a tag may, in one embodiment, be a polypeptide, so that it can be used in a fusion protein and it may come from an organism that is so unrelated to eukaryotic cells that it does not have structural relatives. In a further embodiment, both the tag and the small molecule ligand may not bind cellular proteins. As such, tags suitable for use in the present invention may be derived from chemical reactions, pathways or events that do not normally occur in eukaryotic cells.

The present invention is based upon the finding that the interaction between certain proteins (for example penicillin binding proteins) and compounds comprising β-lactam binding domains (for example β-lactam antibiotic compounds) may be exploited to provide tags or labels suitable for tagging or labelling compounds such as, for example, proteins, peptides, amino acids, nucleic acid (DNA or RNA), small organic molecules, antibodies (or fragments/antigen binding fragments thereof) and carbohydrates. Furthermore, these interactions may provide a means of tagging or labelling compounds such that they can be purified, isolated or extracted from, for example, a solution.

As such, in a first aspect, the present invention provides a use of a beta-lactam (β-lactam) binding protein or fragment, analogue, homologue, variant or derivative thereof, as a tag or label.

In one embodiment, the β-lactam binding protein provided by this invention may be used to tag or label compounds such as proteins, peptides and/or amino acids. It should be understood that while the remainder of this specification discusses fused, tagged or labelled proteins, peptides and/or amino acids, the use of β-lactam binding peptides (or fragments, analogues, homologues, variants or derivatives thereof) may also extend to fused, tagged or labelled nucleic acids, antibodies, carbohydrates and other small organic molecules.

Accordingly, the present invention provides compounds modified to include a β-lactam binding protein, wherein the β-lactam binding protein is a tag or label.

One of skill in this field will be familiar with “protein”, “peptide” and/or “amino acid” tags or labels and it should be understood that these terms encompass any protein, peptide and/or amino acid modified to included a (β-lactam) binding protein—wherein the β-lactam binding protein (or fragment thereof) is a tag or label.

The term “tag” or “label” refers to a moiety which is attached, conjugated, linked or bound to, or associated with, a compound (for example a protein, peptide, amino acid, nucleic acid and/or carbohydrate) and which may be used as a means of, for example, identifying, detecting and/or purifying a compound.

It should be noted that while the prior art may provide examples of tagged and/or labelled β-lactam binding proteins, the tag or label is not the β-lactam binding protein component. In most cases the tag or label is a fluorescent or luminescent moiety such as green fluorescent protein (GFP) or an affinity purification tag such as, for example, poly-His or GST-tags/labels of this type being used to facilitate the detection, identification and/or purification of a β-lactam binding protein. This is distinct from the present invention which provides β-lactam binding peptides for use as compound tags or labels—wherein the β-lactam binding protein is used as a means of detecting, identifying and/or purifying another compound.

In one embodiment, the β-lactam binding peptide may be used to tag or label, for example, recombinant or ectopically expressed proteins, peptides and/or amino acids. In a further embodiment, the β-lactam binding peptides may be used as an affinity tag or label for the purpose of protein purification and the like. One of skill in this field will readily understand that proteins can easily be expressed in cells and that a large number of different cell types may provide suitable expression systems. By way of example, prokaryotic and/or eukaryotic cells, such as, for example, bacterial, fungal, mammalian, plant and/or insect cells, may be exploited as a means of expressing proteins, peptides and/or amino acids.

Proteins, peptides and/or amino acids suitable for tagging or labelling with the β-lactam binding peptides described herein include, for example, mammalian, bacterial, viral, fungal, plant and/or insect proteins, peptides and/or amino acids.

In one embodiment of this invention, the β-lactam binding protein is a penicillin binding protein (PBP) or fragment, analogue, homologue, variant or derivative thereof.

It will readily be understood that PBPs are produced by bacteria, as part of their machinery to synthesise peptidoglycan, a component of their cell wall. Some fungi have evolved to produce inhibitors to these enzymes e.g. penicillin, which covalently bind to the PBPs, preventing the bacteria from building a cell wall and are hence cytotoxic to them. Alexander Fleming discovered penicillin in 1928 and thereby introduced the powerful weaponry of β-lactam-antibiotics in the fight against bacterial pathogens. Penicillin and its derivatives for example ampicillin, amoxicillin and the cephalosporins are covalently bound by the catalytic centre of penicillin binding proteins and act as inhibitors.

Accordingly any PBP may be suitable for use in this invention and as such the term PBP protein should be taken to encompass any type or form of PBP including, for example, the bacterial PBPs 1, 2a, 2b, 3, 4, 5 and 6.

Accordingly, the term “β-lactam binding proteins” as used herein should be taken to include wild-type (i.e. naturally occurring) β-lactam binding proteins as well as any fragments, analogues, homologues, variants or derivatives thereof. In addition, the term “β-lactam binding proteins” may include any of the PBP described herein as well as fragments, analogues, homologues, variants or derivatives thereof.

The term “fragment” should be understood to encompass any part or portion of a β-lactam binding protein. In one embodiment, fragments should retain the ability to bind β-lactam compounds—as such, fragments suitable for use in this invention may include β-lactam binding fragments. Accordingly, β-lactam binding peptide fragments suitable for use in this invention may comprise the β-lactam binding domain of a β-lactam binding peptide.

The term “homologue” may be taken to refer to all members of a particular β-lactam binding protein family or to related β-lactam binding protein. Additionally or alternatively, homologous β-lactam binding protein may include different or equivalent forms of a particular β-lactam binding protein isolated from different species. “homologous” β-lactam binding protein may share a degree of sequence identity/similarity with naturally occurring, or wild-type, β-lactam binding protein. It is well known to one of skill in the art that the quarternary and tertiary structure of a protein/polypeptide is usually highly conserved such that the specific function of that protein/polypeptide is also retained. It is also well known that the primary structure of a protein/peptide may exhibit considerable variation in its sequence without resulting in a significant decrease in the activity of the mature protein/peptide. Consequently a homologous protein/peptide useful in the present invention may share only, for example, 25% amino acid sequence identity with a wild-type β-lactam binding protein when the conserved residues of the two proteins are aligned. Accordingly, homologous β-lactam binding proteins of the present invention may include polypeptides or fragments thereof which show 25%, preferably 40%, more preferably 60% even more preferably 75% and most preferably 90%, 95% or 99% sequence identity or homology with wild-type β-lactam binding proteins.

Variants or derivatives of a particular β-lactam binding protein may include those which have been modified in some way so as to be structurally different. Additionally, or alternatively, a β-lactam binding protein variant may result from the modification, addition and/or removal of one or more substituent moieties and/or amino acids of the primary sequence. The term “variant” or “derivatives” may further include β-lactam binding protein which are structurally distinct from wild-type β-lactam binding proteins.

In one embodiment, variants or derivatives of a particular β-lactam binding protein may include those modified to include one or more additional amino acids, for example at the N-terminus. The additional amino acid(s) may represent sequences which facilitate more efficient mRNA translation and enhance protein stability. This is particularly important when compounds tagged or labelled with β-lactam binding proteins described herein are produced recombinantly in, for example, bacterial cells. By modifying β-lactam binding proteins (or fragments thereof) to include one or more additional amino acid sequences, particularly sequences more readily translated by bacterial cells, it may be possible to improve the yield of recombinantly produced compounds tagged or labelled with β-lactam binding proteins. In one embodiment, the additional amino acid sequence may comprise or consist of poly-His sequences and the like.

It is also to be understood that there are potentially a number of “conservative substitutions” which may occur within the primary sequence of a β-lactam binding protein. By “conservative substitution” it is meant the replacement of an amino acid residue and/or residues with an amino acid residue and/or residues which do not substantially differ in terms of physical and chemical properties from the naturally occurring amino acid residue and/or residues. These “conservative substitutions” will have substantially no effect on the function of the peptide and may yield variant or derivative β-lactam binding protein suitable for use in this invention.

PBP analogues may be synthetically created so as to be functionally and/or structurally homologous, identical or similar to wild-type β-lactam binding proteins.

In one embodiment and by way of example, PBP5, a D-alanyl-D-alanine carboxypeptidase encoded by the E. coli dacA gene, may be suitable for use in the present invention. The amino acid sequence of PBP5 is deposited under Acc No: AP_{—}001281 or NP_{—}415165.1. The PBP5 sequence is provided below.

mntifsarim krlalttalc tafisaahad dlniktmipg
vpqidaesyi lidynsgkvl aegnadvrrd pasltkmmts
yvigqamkag kfketdlvti gndawatgnp vfkgsslmfl
kpgmqvpvsq lirginlqsg ndacvamadf aagsqdafvg
lmnsyvnalg lknthfqtvh gldadgqyss ardmaligqa
lirdvpneys iykekeftfn girqlnrngl lwdnslnvdg
iktghtdkag ynlvasateg qmrlisavmg grtfkgreae
skklltwgfr ffetvnplkv gkefasepvw fgdsdraslg
vdkdvyltip rgrmkdlkas yvinsselha plqknqvvgt
infqldgkti eqrplvvlqe ipegnffgki idyiklmfhh
wfg

As stated, the present invention may utilise whole PBP proteins or fragments, analogues, homologues, variants or derivatives thereof. As such, and with reference to the PBP5 sequence provided above, the PBP protein suitable for use in this invention may comprise the entire PBP5 sequence as shown, or a fragment, analogue, homologue, variant or derivative thereof. By way of example, suitable fragments for use in the methods described herein may comprising residues 37-391 or for example residues 37-297. It should be understood that the N-terminal 36 residues of the abovementioned PBP5 sequence may be removed without substantially affecting the ability of the peptide to bind β-lactam compounds. Furthermore, the C-terminal 106 residues may be removed in order to remove the C-terminal domain, which is not involved in beta-lactam binding and to provide a soluble peptide (Pratt et al., 1986).

Their insolubility and the general assumption that they bind their substrate irreversibly render β-lactam binding proteins such as, PBPs potentially not suitable for use as protein, peptide and/or amino acid (affinity) tags or labels. However, by, for example, modulating their size, solubility and/or by introducing a simple method of reversing the covalent bond, highly effective tags/labels can be generated.

In a second aspect, the present invention provides compounds, for example proteins, peptides and/or amino acids, tagged or labelled with a β-lactam binding protein.

As stated, one of skill in this field will appreciate that both eukaryotic and/or prokaryotic proteins, peptides and/or amino acids including, for example, mammalian, bacterial, plant, viral and/or fungal proteins, peptides and/or amino acids, may be tagged or labelled with any of the β-lactam binding proteins described herein.

It should be understood that the tagged or labelled proteins, peptides and/or amino acids, particularly recombinant proteins or peptides may be fused, bound or otherwise associated directly with, or to, any of the β-lactam binding proteins described herein. Alternatively, tagged or labelled proteins, peptides and/or amino acids may be fused, bound or otherwise associated indirectly with, or to, any of the β-lactam binding proteins described herein, via some linker moiety. For example suitable linker moieties may include short amino acid sequences comprising, for example one or more amino acids.

In one embodiment, the invention relates to fusion proteins/peptides, for example isolated fusion proteins/peptides, comprising or consisting essentially of a β-lactam binding protein (or fragment, analogue, homologue, variant or derivative thereof) and a heterologous protein/peptide or amino acid(s).

In a third aspect, the present invention provides a method of generating a protein or peptide tagged or labelled with a β-lactam binding protein (for example a fusion protein), said method comprising the steps of cloning a nucleic acid sequence encoding a protein or peptide to be tagged or labelled into a vector comprising a nucleic acid sequence encoding a beta-lactam (β-lactam) binding protein according to this invention. By using vectors which are capable of directing the expression of particular nucleic acid sequences in, for example, bacterial, fungal, mammalian and/or insect cells and cloning the nucleic acid sequence of the protein or peptide to be tagged, upstream or downstream of the sequence which encodes the beta-lactam (β-lactam) binding protein (and optionally a linker moiety), it is possible to generate a protein or peptide fused either directly (i.e. without the use of a linker moiety) or indirectly (i.e. with use of a linker moiety) to a β-lactam binding protein.

Accordingly, a fourth aspect of this invention provides a vector, preferably an expression vector, comprising a nucleic acid sequence encoding any of the β-lactam binding proteins described herein (including, fragments, variants, analogues or derivatives thereof) for the expression/production of proteins, peptides and amino acids tagged or labelled with a β-lactam binding protein (or fragment thereof). In one embodiment, the vectors provided by this invention may further comprise one or more sites into which a nucleic acid sequence encoding a protein or peptide to be fused to or tagged/labelled with said β-lactam binding protein (or fragment, derivative, variant or analogue thereof) may be introduced or “cloned”. Such sites may comprise one or more sites cleavable with the use of restriction enzymes.

In addition, one of skill in this field will recognise that expression vectors suitable for use in this aspect of the invention may further comprise one or more promoter sequences capable of directing expression in prokaryotic or eukaryotic cells such as, for example, mammalian, fungal, bacterial, plant and/or insect cells.

In a further aspect, the present invention provides host cells transfected or transformed with a vector as described herein. Eukaryotic or prokaryotic cells, such as, for example, plant, insect, mammalian, fungal and/or bacterial cells, may be transfected with one or more of the vectors described herein. One of skill in this field will be familiar with the techniques used to introduce heterologous or foreign nucleic acid sequences, such as expression vectors, into cells and these may include, for example, heat-shock treatment, use of one or more chemicals to induce transformation/transfection, the use of viral carriers and/or techniques such as electroporation.

In view of the above, a sixth aspect of this invention provides a method of producing or generating a protein or peptide fused to (i.e. tagged or labelled with) a beta-lactam (β-lactam) binding protein, said method comprising the steps of introducing into a cell a nucleic acid sequence encoding a protein or peptide fused to a beta-lactam (β-lactam) binding protein and maintaining the cell under conditions suitable to permit or induce expression of the fused protein or peptide. In one embodiment, the nucleic acid is introduced into the cell in the form of an expression vector.

The protein tags or labels provided by this invention (i.e. the β-lactam binding proteins and/or fragments, derivatives, variants, homologues and/or analogues thereof) may find particular application as affinity tags or labels which may be used in affinity purification and/or chromatography techniques for the purification, isolation and/or removal of certain proteins from, for example, solution; particularly solutions containing many different types of peptide and/or protein.

Thus, in a seventh aspect, the present invention provides a method of purifying, isolating and/or extracting compounds, said method comprising the steps of:

(a) contacting a sample comprising tagged or labelled compounds according to a second aspect of this invention, with a compound capable of binding the β-lactam binding protein tag or label;

(b) removing any unbound compound; and

(c) separating the bound tagged or labelled protein from the compound capable of binding the β-lactam binding protein tag or label.

It should be understood that the method provided by this aspect of the invention may be used to purify large quantities of a particular protein and/or or to selectively purify, isolate and/or remove a particular protein or peptide from a solution. The method may also be used to purify, isolate or remove protein or peptides from heterogeneous protein populations—particularly solutions comprising heterogeneous protein populations. As such, the term “compound” as used in the seventh aspect of this invention, may refer to proteins, peptides and/or amino acids as well as nucleic acids, antibodies, carbohydrates and other small organic molecules.

In addition, one of skill in this field will appreciate that it should be understood that a compound capable of binding a β-lactam binding protein tag or label may otherwise be referred to as a “ligand” for the β-lactam binding protein.

Typically the compound capable of binding the β-lactam binding protein tag or label is complexed to (coupled, immobilised or otherwise associated with, or to) an appropriate support substrate, for example a solid support. Advantageously the compound may be complexed to, said solid support by, for example, covalent, ionic or hydrophobic interactions. It should be understood that hereinafter, references to a “support substrate” include substrates which are complexed to compounds capable of binding β-lactam binding proteins.

In one embodiment, the support may, for example, comprise agarose, sepharose, polyacrylamide, agarose/polyacrylamide co-polymers, dextran, cellulose, polypropylene, polycarbonate, nitrocellulose, glass paper or any other substance capable of providing a suitable solid support. Additionally, or alternatively supports such as magnetic particles, may also be used.

Advantageously the support substrate may be in the form of granules, a powder or a gel suitable for use in chromatography such as those available from GE-Healthcare, Sigma-Aldrich, Expedeon and others.

In one embodiment, the support substrate may be packed into a column adapted to receive a sample comprising compounds, for example proteins, particularly proteins (or peptides) tagged or labelled with a β-lactam binding protein as described herein. Advantageously, via gravity or the action of a pump, the sample may be added to the column and allowed to pass through and/or over the support substrate. In this way any compound (for example a protein, peptide or amino acid), comprising a β-lactam binding tag or label may bind to the support substrate and may be retained in the column, while all other, non tagged or labelled compounds will pass through.

In one embodiment, the compound capable of binding the β-lactam binding protein tag or label of the protein to be purified, isolated and/or extracted, is a β-lactam compound—i.e. a compound comprising a β-lactam ring. By way of example, the compound capable of binding the β-lactam binding protein tag or label may comprise any β-lactam compound such as, for example, penicillin, ampicillin, amoxicillin or derivatives, variants, analogues or homologues thereof, but also antibiotics of the cephalosporin group of β-lactam compounds. In any case it will be advantageous, if the compound capable of binding the β-lactam binding protein tag or label does not bind other cellular proteins.

In one embodiment, suitable derivative or variant compounds capable of binding the β-lactam binding protein tag or label include, for example, isopenicllin and/or cyclobutane derivatives of penicillin.

In other embodiments, the compound capable of binding the β-lactam binding protein tag or label is an antibody selective, specific or exhibiting affinity for a β-lactam binding protein. In certain embodiments, the antibody may be a monoclonal and/or polyclonal antibody. Furthermore, the term “antibody” should be taken to include antigen or epitope binding fragments thereof. The term “fragment may include, for example, Fab, Fab₂, Fab₃, V_{H}, V_{L} domains or fragments as well as, for example, minibodies, dia/tria and tetrabodies. Antibodies capable of binding β-lactam binding proteins may be immobilised to any of the substrates described herein.

The method provided by the seventh aspect of this invention represents a significant advantage over prior art methods. In particular, the compounds capable of binding the β-lactam binding protein have been shown to be highly selective and, in contrast to certain prior art systems (for example those utilising GST tags or labels) the amount of non-specific protein bound to the compounds capable of binding the β-lactam binding protein is very low. In one embodiment and when compared to techniques which utilise, for example, GSH-sepharose, the total amount of protein bound non-specifically to, for example ampicillin-sepharose (i.e. a compound capable of binding a β-lactam binding protein (ampicillin) immobilised onto a support substrate (sepharose)), is approximately 50 times less.

In order to capture tagged or labelled compounds (for example proteins or solutions comprising proteins/peptides), including those tagged or labelled with the PBPs described herein, tagged or labelled compounds may be incubated or contacted with a compound capable of binding a β-lactam binding protein tag or label at an ambient temperature. For example, the solution may be incubated or contacted with a compound capable of binding a β-lactam binding protein tag or label at an ambient temperature of approximately 15° C.-37° C., approximately 18° C.-25° C. or approximately 20° C.-23° C. In one embodiment the ambient temperature may be approximately 22° C. Under these conditions, compounds, for example, proteins/peptides and/or amino acids, tagged or labelled with a PBP described herein may bind to compounds capable of binding a β-lactam binding protein tag or label.

Binding between a tagged or labelled compounds of this invention and a compound capable of binding a β-lactam binding protein tag or label, may occur rapidly at ambient temperatures and may be complete within about 30 minutes. It should be understood that when compounds capable of binding β-lactam binding protein tags or labels are used to capture or bind compounds, for example proteins or peptides, tagged or labelled with a PBP, the precise length of incubation/contact time may vary depending on, for example, the concentration of the tagged compound in the solution and the concentration of contaminants.

Generally, when purifying a particular compound such as, for example a protein or peptide, proteinaceous material/compounds not bound to a compound capable of binding a β-lactam binding protein, is removed before bound material is released. Where the compound is complexed with a support substrate, the support substrate may be washed, for example, with an osmotically balanced and/or neutral solution or buffer such as e.g. Tris, MOPS, HEPES or phosphate buffer and/or NaCl solution (for example a mild NaCl solution (0.15M) or stringent NaCl buffer (1M). Typically such a procedure is repeated a number of times to maximise the amount of unbound, contaminating material removed from the support substrate.

The inventor has discovered that in order to release material (i.e. tagged or labelled proteins, peptides or amino acids according to this invention) bound to a compound capable of binding a β-lactam binding protein, bound material/compound capable of binding β-lactam binding protein complexes may be incubated or contacted with any suitable cold elution buffer. It should be understood that the term “cold” may refer to elution buffers which are at a temperature of between 1° C. and 10° C., typically 2° C. and 8° C. and in some embodiments at approximately 4° C. Cold temperatures such as these may be achieved by incubating elution buffers “on ice” for prolonged period of time. After a suitable period of incubation/contact with a cold elution buffer, β-lactam binding peptide tagged or labelled compounds, for example proteins, peptides or amino acids, bound to compounds capable of binding β-lactam binding proteins (which may optionally be complexed to some form of substrate) may be eluted therefrom. Additionally, and in order to accelerate the elution procedure, elution buffers may be supplemented with compounds comprising hydroxyl groups. Suitable compounds comprising hydroxyl groups may include, for example, alcohols such as glycerol. Sugars, for example monosaccharides, such as glucose or fructose or disaccharides such as saccharose (table sugar) are also effective in accelerating elution. Additionally or alternatively, ionic agents, such as, for example, 100 mM NaCl and/or detergents, such as low concentrations of, for example, 0.05%-1% Trition X-100 or 0.01%-0.5% Brij35 may also be used to optimize recovery—it is well known that these reagents minimise unspecific binding of protein to some matrices, such as sepharose.

For example an ice cold elution buffer may be supplemented with a glycerol solution comprising approximately 1% to 50%, 2% to 40%, 3% to 30%, 4% to 20%, 5%-10% and preferably 5% glycerol. Elution solutions of this type may be added to a support substrate comprising a compound capable of binding a PBP and incubated at, approximately 0-5° C. for a period of about 2 minutes to about 20 hours.

As with washes to remove unbound material, the yield of purified compounds, for example tagged or labelled proteins, peptides and/or amino acids provided by this invention, may be further increased by repeating the application of elution buffers.

The “eluate”, (predominantly comprising, for example, proteins/peptides/amino acids tagged or labelled with a β-lactam binding peptide) released from a compound capable of binding a β-lactam binding protein or a support substrate, may be collected or removed in a number of ways. In one embodiment and where the compound capable of binding a β-lactam binding peptide is complexed to a support substrate and packed into a column, the eluate may be collected via the action of gravity as a suitable elution solution passes through and over the support matrix. In other embodiments, the eluate may be removed from support substrates by means of a pump or, alternatively by means of centrifugation.

The use of centrifugation is particularly desirable where the support substrate is contained within a column suitable for placement in a microfuge or the like—such column may be known in the art as “spin columns”.

Without wishing to be bound by theory, the inventors hypothesise that upon binding, a specific serine residue of the PBP acylates the β-lactam antibiotic. However, the PBP also catalyses deacylation, which is not normally observed, as acylation resulting in covalent binding, occurs much faster, so the PBP appears irreversibly bound to its β-lactam substrate. At reduced temperatures, for example any of the “cold” temperatures described above, Penicillin Binding Proteins detach from the β-lactam substrates and may be unable to bind again. As such the use of cold or cool temperatures facilitates elution. With regards the use of supplemental factors to further accelerate the elution procedure, without wishing to be bound by theory, the inventor hypothesises that addition of glycerol or sugar compounds either slows the rate of acylation or (more likely) increases the level of deacylation—a theory which is supported by the fact that unlike other affinity purification systems, elution of material bound to the support substrates described herein is not brought about by flooding the system with soluble ligand (i.e. β-lactam compound).

At ambient temperatures, for example 15° C.-37° C., β-lactam binding peptides, such as PBPs, bind rapidly, stoichiometrically and covalently to their ligands, for example, a β-lactam compound such as ampicillin—even when the ligand is bound, immobilised or associated with a support substrate such as sepharose. Nevertheless, at cold temperatures and under the mild buffer conditions described above the β-lactam binding peptide tag or label can easily be released from its ligand and recovered in a solution eluted therefrom. Furthermore, it will be appreciated that compounds, for example, proteins eluted from the support substrate using the buffers and conditions described herein, are compatible with a range of further techniques, such as assays or methods involving enzymes and analytical or immunological methods involving SDS-page or Western Blotting.

In one embodiment, the method of isolating, extracting and/or purifying compounds, for example, proteins or peptides may further comprise the steps of first maintaining a host cell according to the fifth aspect of this invention under conditions suitable to induce the expression of a compound, for example a protein/peptide or amino acid fused to (i.e. tagged or labelled with) a β-lactam binding peptide—such as those listed according to the second aspect of this invention. Thereafter, the tagged or labelled compound may be contacted with a compound capable of binding the β-lactam binding protein tag or label and subjected to the methodology outlined in the seventh aspect of this invention.

In a further aspect, the present invention provides means of detecting compounds tagged or labelled with a β-lactam binding protein. In one embodiment the detection means may take the form of antibodies, for example, monoclonal and/or polyclonal antibodies specific, selective or exhibiting affinity for a compound tagged or labelled with a β-lactam binding peptide. One of skill will appreciate that the epitope(s) recognised by antibodies of this type may sit within the boundary between the compound and the β-lactam binding peptide tag or label. It should be understood antibody fragments capable of binding compounds tagged or labelled with β-lactam binding peptides are also within the scope of this invention.

One of skill will appreciate that any of the antibodies described herein (including those described as being useful in the seventh aspect of this invention) may be used to detect or screen for, compounds which have been tagged or labelled with a β-lactam binding protein.

In other embodiments, tagged or labelled, for example fluorescently/chemiluminescently and/or luminescently tagged or labelled β-lactam compounds (such as those described herein) may be used to detect or screen for, compounds which have been tagged or labelled with a β-lactam binding protein.

In a yet further aspect, the present invention provides a kit for the production/purification of compounds tagged or labelled with the β-lactam binding proteins described herein, said kit comprising a vector as described in the fourth aspect of this invention. In one embodiment, the kit provided by this invention further comprises instructions for use.

In other embodiments, the kit may comprise cells, advantageously competent cells, into which the vector may be transferred/transfected. In one embodiment the cells may be E. coli cells competent for transformation with a vector.

In other embodiments, the kit comprises buffers and other reagents for use in transformation/transfection protocols.

Additionally, or alternatively, the kits provided by this invention may further comprise compounds capable of binding β-lactam binding proteins as described herein (for example β-lactam compounds and/or antibodies capable of binding β-lactam binding proteins), optionally coupled to support substrates, for use in purifying compounds tagged or labelled with β-lactam binding proteins and expressed by transformed/transfected cells and/or for use in identifying and/or detecting tagged or labelled compounds.

In one embodiment, the support substrate may be provided in the form of a micro-spin column for use in a microfuge.

The invention will now be described in detail with reference to the following Figures which show:

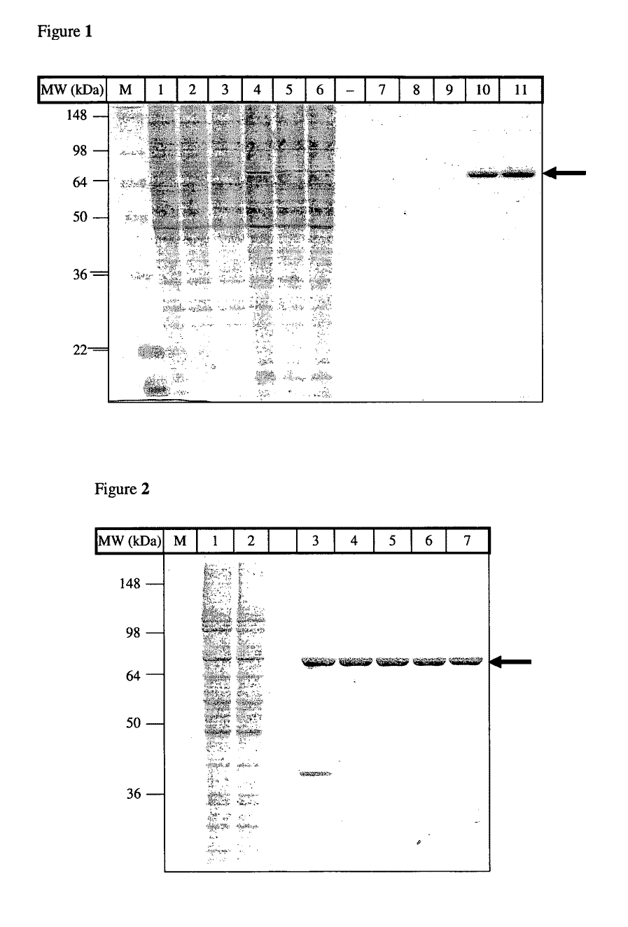

FIG. 1: Purification of dac-tagged green fluorescent protein (GFP) from transfected HEK293 cells. It should be understood that GFP serves as an example for any protein of interest and is not an integral part of the invention. Five 10 cm dishes of HEK293 cells were transiently transfected with a vector encoding for green fluorescent protein (GFP) (lanes 1, 2, 3, 7 and 9) or a vector encoding for GFP, which is N-terminally tagged with e. coli PBP5, the gene product from the dac A gene (aa 37-392 follow by a Prescission Protease cleavage site) (lanes 4, 5, 6, 8, 10 and 11). The cells were lysed in 40 mM Tris pH 7.5, 0.1% Triton X-100, 1 mM EDTA, 1 mM EGTA, protease inhibitors and insoluble matter was removed by centrifugation for 5 min at 15000×g. 5 μg of the extract was separated before (lanes 1 and 4) or after a pulldown with Tris-sepharose (lanes 2 and 5) or with ampicillin-sepharose (lanes 3 and 6) respectively.

Amounts (0.5 mg) of cell extract were incubated at 22° C. for 1 hour with 25 μl Tris-sepharose (lanes 7 and 8) or ampicillin sepharose (lanes 9 and 10). The sepharose was collected and washed 5 times in 40 mM Tris, 1M NaCl, 0.1% Triton X-100 and once with 40 mM Tris-buffer 22° C. The sepharose was transferred to spin filter units (Amicon, 0.45 μm PES membrane), spun dry and then incubated for 16 h at 4° C. with 24±1 of 40 mM Tris, 5% glycerol, 0.1M NaCl, 0.01% Triton X-100, 7 mM 2-mercaptoethanol (lanes 7-10). The eluates were collected by centrifugation and the sepharose sediments were incubated with another 20 μl of the same buffer for 30 min. The sediments were spun dry again and the eluates from the first and second spin were pooled in order to maximise recovery. The extract from lane 8, which, after incubation with Tris-sepharose still contained all the dac-tagged GFP, was incubated with ampicillin-sepharose, washed and eluted as described above, with the only difference that the elution buffer also contained 10 mM ampicillin (lane 11).

These eluates (45 μl) were mixed with 15 μl 4×SDS-loading buffer (320 mM Tris pH 6.8, 8% SDS, 32% glycerol, 1.14 M β-Mercapto-EtOH, 0.01% Bromphenolblue), denatured by 3 min incubation at 95° C. and separated by SDS-PAGE on a 10% gel. The gel was stained with colloidal coomassie and destained extensively in water. The gel was scanned and the image saved as a jpeg file. The 75 kDa protein in lanes 10 and 11, indicated by a small arrow, represents the dag-tagged GFP fusion protein (validated by Mass-Spectrometry Fingerprinting). Numerous other proteins contaminate the sample each in very small amounts. Most of these proteins contaminate the sample due to unspecific binding to sepharose, rather than the ampicillin ligand, with the exception of a protein at 45 kDa (dac a tag) and 25 kDa (GFP), a result of cleavage between the tag and the GFP by an unknown protease. Clathrin heavy chain (CHC) binds specifically to ampicillin sepharose and can be eluted in the presence of ampicillin. It presents as a minor high MW contaminant in the eluates.

FIG. 2: Effect of various NaCl concentrations in the washing buffer. Ten 10 cm dishes of HEK293 cells were transiently transfected with a vector encoding for GFP, which is N-terminally tagged with e. coli PBP5, the gene product from the dac A gene (aa 37-392 follow by a Precision Protease cleavage site). The cells were lysed in 40 mM Tris pH 7.5, 0.1% Triton X-100, 1 mM EDTA, 1 mM EGTA, protease inhibitors and insoluble matter was removed by centrifugation for 5 min at 15000×g. 5 μg of the extract was separated before (lane 1) or after a pulldown with ampicillin-sepharose (lane 2) respectively.

Amounts (2.5 mg) of cell extract were incubated for 45 min at 22° C. with 125 μl ampicillin-sepharose. The sepharose was collected and the sediment distributed into 5 vials. The sediments were washed 5 times at 22° C. in 40 mM Tris, 0.1% Triton X-100 with different NaCl concentrations for each aliquot (lane 3=no NaCl, lane 4=0.125 M, lane 5=0.25 M, lane 6=0.5 M and lane 7 with 1M NaCl). The sediments were then washed once with 40 mM Tris-buffer and transferred to spin filter units, spun dry and then incubated for 8 h at 4° C. with 25 μl of 40 mM Tris, 5% glycerol, 0.1M NaCl, 0.01% Triton X-100, 7 mM 2-mercaptoethanol (lanes 3-7). The eluate was collected, separated on SDS-PAGE, analysed and presented as described for FIG. 1.

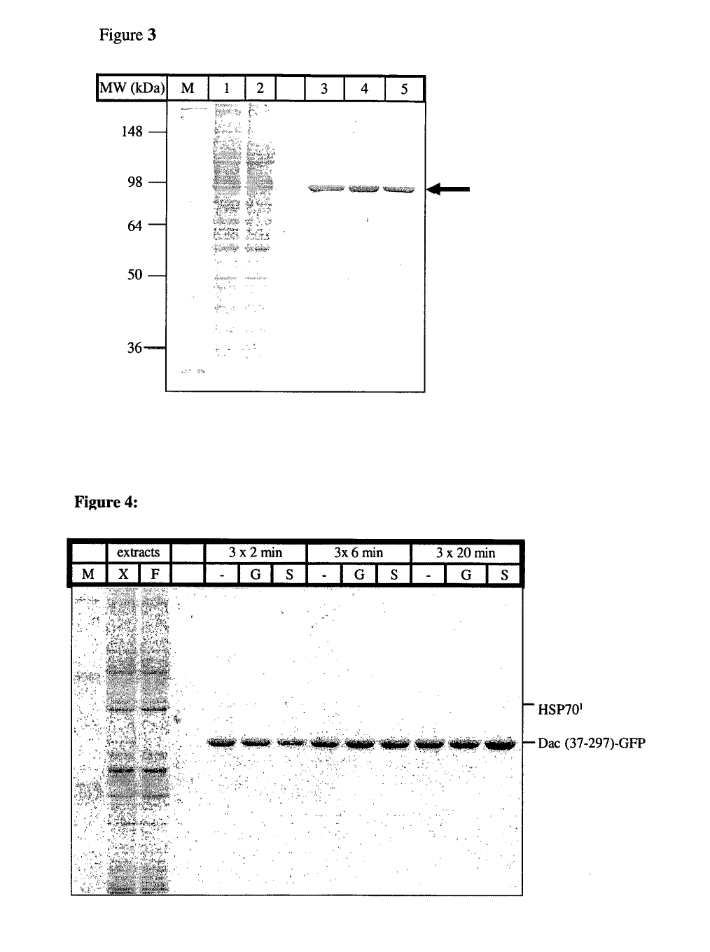

FIG. 3: Effect of ampicillin in the washing buffer. Five 10 cm dishes of HEK293 cells were transiently transfected with a vector encoding for dac-tagged GFP and lysed as described for FIG. 1. 5 μg of the extract was separated before (lane 1) or after a pulldown with ampicillin-sepharose (lane 2) respectively. 1.5 mg cell extract was incubated for 45 min at 22° C. with 120 μl ampicillin-sepharose. The sepharose was collected and the sediment distributed into 3 vials. The sediments were washed 3 times in 40 mM Tris, 0.1% Triton X-100, 0.15M NaCl and then twice in the same buffer either without ampicillin (lane 3) or supplemented with 10 mM (lane 4) or 30 mM ampicillin (lane 5). The sediments were then washed twice with 40 mM Tris-buffer to remove NaCl and ampicillin and transferred to spin filter units (Amicon, 0.45 μm PES membrane), spun dry and then incubated for 8 h at 4° C. with 25 μl of 40 mM Tris, 5% glycerol, 0.1M NaCl, 0.01% Triton X-100, 7 mM 2-mercaptoethanol (lanes 3-5). The eluates were collected and analysed as described for FIG. 1. The purified recombinant dac-A-GFP fusion protein is indicated with an arrow.

FIG. 4: PBP5 aa 37-297 elution conditions. Ten 10 cm dishes of HEK293 cells were transiently transfected with a vector encoding for PBP5 (aa 37-297) followed by GFP. Three days after transfection the cells were lysed and the lysate was clarified by centrifugation. 20 mg of the extract was incubated for 30 min at ambient temperature with 200 μl of Ampicillin sepharose, which had been prepared from Ampicillin and NHS-activated sepharose. The extract was removed and the sepharose washed five times with a buffer containing 0.5M NaCl and once in a buffer containing no NaCl. The sepharose was aliquoted into 9 spin filters and incubated for 3×2 minutes, 3×6 minutes or 3×20 min with 20 μl elution buffer (30 mM Tris pH 7.5, 100 mM NaCl, 0.03% Brij 35, 14 mM 2-mercaptoethanol). In lanes (G) this buffer had been supplemented with 5% glycerol and in lanes (S) the buffer had been supplemented with 180 mM saccharose). The purified fusion protein is indicated with an arrow.

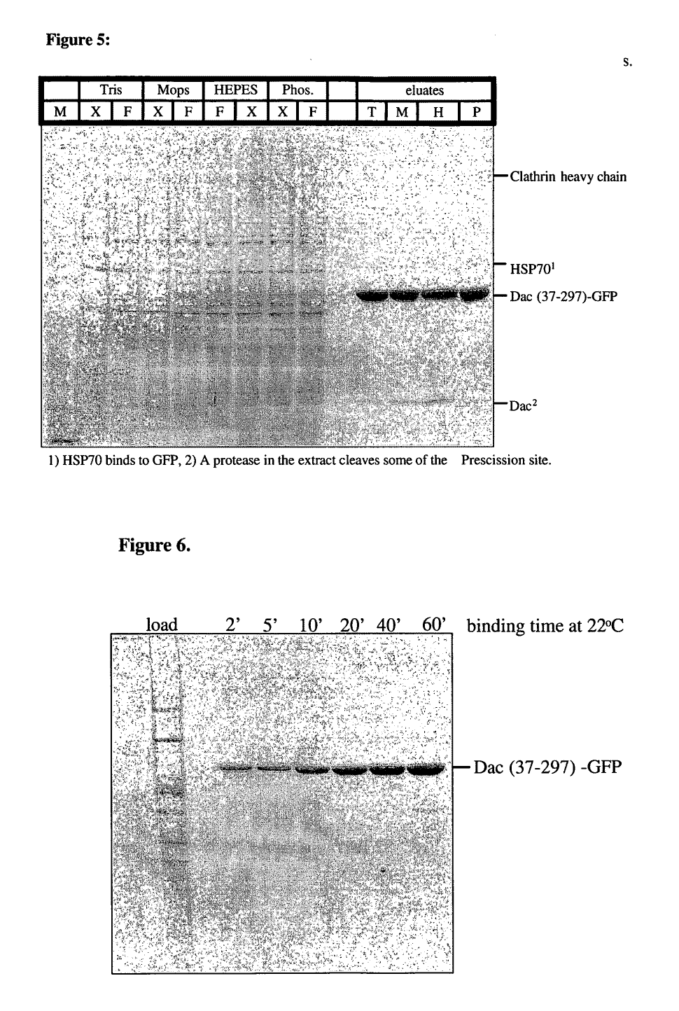

FIG. 5: Purification of the catalytic domain of E. coli PBP5 fused to GFP in various buffer systems. Five 10 cm dishes of HEK293 cells were transiently transfected with a vector encoding for the catalytic domain of dac A aa 37-297 followed by a Prescission Protease site and GFP. The cells were collected and split into 4 aliquots. The cells were lysed at ambient temperature in 1 ml of 0.2% Triton X100, 1 mM EDTA, 1 mM EGTA, protease inhibitors and either 30 mM Tris pH 7.5 (T), 30 mM MOPS pH 7.5 (M), 30 mM HEPES pH 7.5 (H) or 30 mM Phosphate pH 7.5 (P). Insoluble material was sedimented by centrifugation and the lysates were incubated at ambient temperature for 30 min with 20 μl equilibrated ampicillin sepharose, which had been prepared from NHS-activated sepharose. The sediments were washed at ambient temperature with washbuffers containing 250 mM NaCl, 0.2% Triton X100 and either 30 mM Tris pH7.5 (T), 30 mM MOPS pH 7.5 (M), 30 mM HEPES pH 7.5 (H) or 30 mM Phosphate pH 7.5 (P). The sepharose sediments were then transferred to spin filter units and incubated on ice with 20 μl elution buffer containing 100 mM NaCl, 5% glycerol, 0.03% Brij35 and either or 30 mM Tris pH7.5 (T), 30 mM MOPS pH 7.5 (M), 30 mM HEPES pH 7.5 (H) or 30 mM Phosphate pH 7.5 (P). The eluate was collected by centrifugation and the elution process was repeated twice. The eluates were analysed by SDS-PAGE and Coomassie Blue staining as described above. Lanes labeled with X have been loaded with 5 μg extract. Lanes labeled with F have been loaded with the depleted extract that is the flowthrough.

FIG. 6: Binding kinetic of PBP5 aa 37-297-GFP to ampicillin sepharose Ten 10 cm dishes of HEK293 cells were transiently transfected with a vector encoding for dac-tagged GFP (aa 37-297) and lysed as described above. 5 μg of the extract was separated (load). 2.5 mg cell extract was incubated at ambient temperature for 2, 5, 10, 20, 40 or 60 min (as indicated) with 20 μl ampicillin-sepharose. The sepharose was collected and the sediment distributed into 6 vials. The sediments were washed 5 times in 40 mM Tris, 0.03% Brij 35, 0.25M NaCl and then twice with 40 mM Tris-buffer and transferred to spin filter units (Amicon, 0.45 μm PES membrane), spun dry and then incubated for 20 min on ice with 20 μl of 40 mM Tris, 5% glycerol, 0.1M NaCl, 0.01% Triton X-100, 7 mM 2-mercaptoethanol. The eluates were collected and analysed as described for FIGS. 1 and 2. The purified recombinant dac-A-GFP fusion protein is indicated with an arrow.

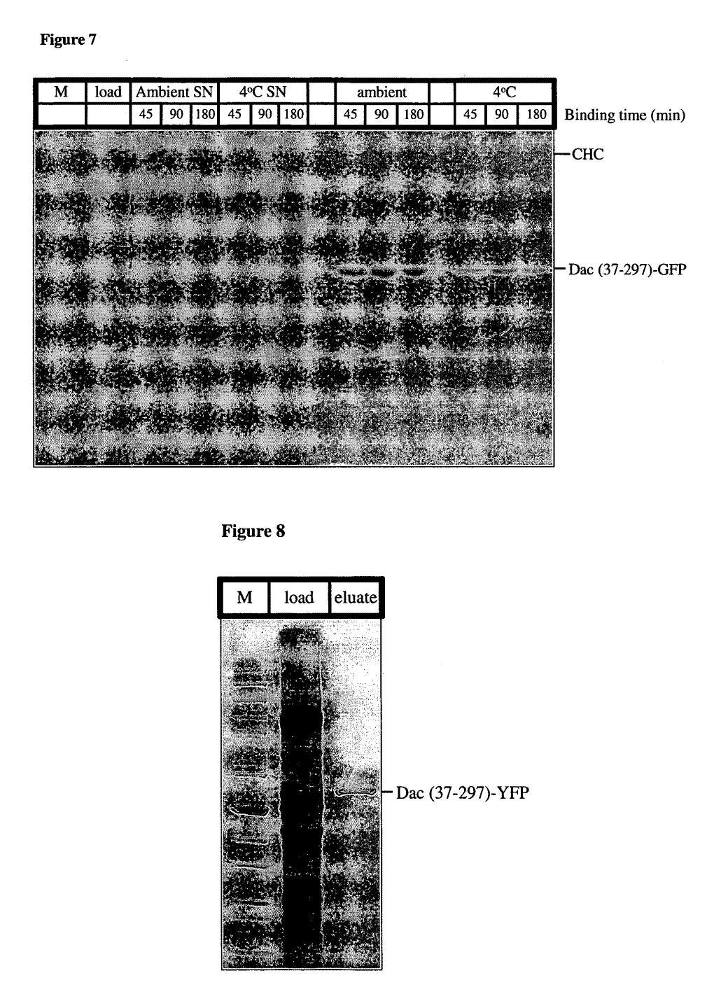

FIG. 7: Effect of incubation temperature on yield. Ten 10 cm dishes of HEK293 cells were transiently transfected with a vector encoding for E. coli PBP5 (aa 37-297) followed by GFP and lysed as described above. 2.5 mg cell extract was incubated for 45, 90 or 180 min with 20 μl ampicillin-sepharose at ambient temperature or at 4° C. as indicated. The sediments were washed 5 times in 40 mM Tris, 0.03% Brij 35, 0.5M NaCl. The sediments were then washed twice with 40 mM Tris-buffer and transferred to spin filter units (Amicon, 0.45 μm PES membrane), spun dry and then eluted by incubation with 20 μl of 40 mM Tris, 5% glycerol, 0.1M NaCl, 0.01% Triton X-100, 7 mM 2-mercaptoethanol. Elution was repeated twice, so that the combined elution volume was 60 μl. 5 μg of the extract (load) and the supernatants (SN) and all of the eluates were separated on a 10% SDS-gel and analysed as described above.

FIG. 8: The dac-tag in Dictostelium discordium. E. coli PBP5 (aa37-297) followed by yellow fluorescent protein was cloned into a Dictostelium expression vector. Dictostelium cells were stably transfected with this plasmid and when the cells became fluorescent the PBP5-YFP fusion protein was purified using ampicillin sepharose and the method described above.

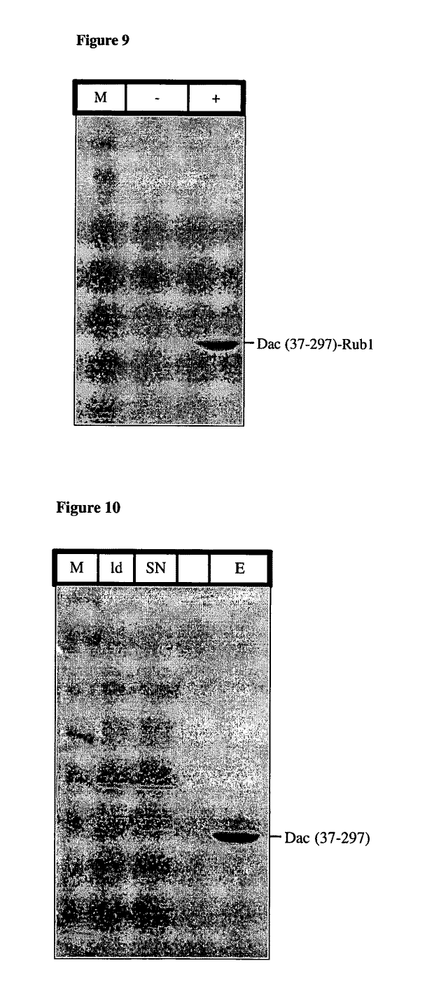

FIG. 9: The dac-tag in Saccharomyces cerevisiae. E. coli PBP5 (aa37-297) followed by the Rub 1 protein was cloned into an inducible S. cerevisiae expression vector. S. cerevisiae were transformed with this plasmid and left either uninduced (−) or were induced with galactose (+) to facilitate expression of the PBP5-Rub1 fusion protein. The fusion protein was purified using ampicillin sepharose as described above.

FIG. 10: The dac tag in E. coli. BL21 cells were transformed with a plasmid containing PBP5 aa 37-297 under the control of the viral T7 promoter. Cells were grown to OD=0.6 and induced with 0.2 mM IPTG. The cells were further incubated at 37° C. for 4 h and collected by centrifugation. The cells were resuspended in 40 mM Tris pH 7.5, 1 mM EDTA, 1 mM EGTA, 10 mg/ml Leupeptin, 1 mM Pefabloc and lysed by sonication. Insoluble material was sedimented by centrifugation and the extract was incubated with ampicillin sepharose at ambient temperature for 1 h. The extract was removed and the sepharose was washed. The protein was collected and analysed as described above.

### MATERIAL AND METHODS

### Cloning of the dac A Constructs

The dac-A fragment Met37-Asp 392 was cloned by PCR using the forward primer ATCGCTAGCCACCATGATCCCGGGTGTACCGC and the reverse primer GTAAGCTTGGGCCCCTGGAACAGAACTTCCAGATCAATGATTTTGCCGAA GAAGTTACC. A site for PreScission protease was added, followed by a multicloning site. The PCR fragment was cloned into Nhe1-HINDIII sites of the pEGFP-N1 vector. The insert was subcloned into various expression vectors, such as pEGFP for expression in human cells and pET24 and pET28a for bacterial expression.

### Cell Culture and Transfections

HEK293 cells were grown in Dulbeccos Modified Eagles Medium (DMEM), supplemented with 10% fetal calf serum at 37° C. in an atmosphere containing 5% CO₂. 10 cm dishes cells were transfected using the Calcium Phosphate method. Briefly, for each dish of cells 5-15 μg DNA was mixed with 61 μl 2 M CaCl and made 500 μl with H₂O. Then 500 μl 2×HBS (50 mM HEPES pH 7.4, 280 mM NaCl, 1.5 mM Na₂HPO₄×2 H₂O) was added dropwise whereby the DNA mix was constantly vortexed. This mix was then carefully dropped onto the dishes and they were left to become transfected overnight. The culture medium was replaced the next morning and the cells were left another 2-3 days before being carefully washed and collected in PBS.

### Preparation of Ampicillin Affinity Sepharose.

Activated CH-sepharose or NHS-activated sepharose was swollen in 1 mM HCl for 20 min and then thoroughly washed with 40 volumes 1 mM HCl. The sepharose was mixed with an equal volume of 0.2M ampicillin in H₂O and incubated for 3 hours at 15° C. or for 90 min at 22° C. The ampicillin was washed away with 15 volumes of 0.1 M NaHCO₃, 0.25M NaCl pH 8.0. The sepharose was mixed with 0.1M Tris pH 8.0 and incubated for 1 hour. The sepharose was washed with 20 volumes 0.1M Na-acetate pH 4.5, followed by 20 volumes 0.1M Tris pH 8.0. The sepharose was washed with 20 volumes 20% EtOH, and stored in 20% EtOH. Ampicillin sepharose made from NHS-activated sepharose has a much better binding capacity compared to Ampicillin sepharose made from CH-activated sepharose.

### Preparation of Cell Extracts, Affinity Purification and Analysis.

Cell sediments were resuspend well in 5-10 volumes lysisbuffer (10-50 mM Tris pH 7.0, 7.5 or 8.2, 0.1%-1% Triton X-100, 1 mM EDTA, 1 mM EGTA, 1 mM Pefabloc®, 10-40 μg/ml Leupeptin) incubated for 5 minutes on ice and then clarified by centrifugation for 5 min at 15000×g at 4° C. Other buffer systems such as HEPES, phosphate buffer or MOPS may be used as well as shown in FIG. 6. Ampicillin sepharose was washed three times in 10 volumes of H₂O and made a 50% v/v slurry. Binding to ampicillin sepharose is fast and efficient at ambient temperature such as 22° C., but very inefficient at colder temperatures, such as, for example, 4° C. The ampicillin sepharose was added to the cell extract with a ratio of 10 μl-200 μl sepharose (20-400 μl slurry) per ml cell extract. The extract was agitated in a tube roller or rocker shaker for 2 min to 3 h to allow the tagged proteins to bind to the ligand. An incubation time of 20-40 min is most effective, whereas very short or very long incubation may reduce yield. The sepharose was sedimented by centrifugation or collected in an empty column e.g. Biorad econopac column or a spin filter. The sepharose was washed thoroughly with 10-40 mM Tris pH 7.5, 0-1 M NaCl, 0.1%-1% Triton X-100. Other mild detergents such as 0.03% Brij35 may be used. Other buffers may be used, Small sediments (<50 μl) in 1.5 ml reaction tubes may be washed 4-5 times with 1 ml buffer, using vortexing and centrifugation. Larger sediments may be washed with >50 volumes buffer using columns and possibly a pump. The sepharose was then washed in 10 volumes 40 mM Tris pH 7.5 to reduce the concentration of Triton and NaCl. Proteins were then eluted by incubating the sepharose with 40 mM Tris pH 7.5, 0.1 M NaCl, 0.02% Triton, 5% glycerol for 5 min to 20 hours at 4° C. or on ice, followed by collection of the eluate. Elution on ice works best. Beside glycerol other hydroxyl containing substances, such as sucrose can accelerate elution. The tag does not elute efficiently at ambient temperature. Ambient temperatures may be exploited to digest the protein of interest with a suitable protease such as Thrombin, TEV or Rhino protease 3C (Prescission Protease), so that it may be recovered without the tag. With smaller samples, for example for proteomics purposes, it is useful to collect by centrifugation utilising a spinfilter for example with a 0.45 μM membrane, in order to achieve high protein concentrations and maximize sample purity. This will dry out the sepharose and leave little protein on it. This will also prevent the contamination of the eluate with sepharose, an important aspect with regards to high sample purity. The proteins were analysed by denaturation and SDS-polyacrylamide gel electrophoresis. However, other applications, such as enzyme assays or other methods of analysis may be used.

### Discussion

The experiment detailed in FIG. 1 shows that a fragment of E. coli penicillin binding protein 5, the gene product of the e. coli gene dac A, can be used in combination with ampicillin affinity media to purify a protein of interest—here, for demonstration purposes, green fluorescent protein with great purity in a simple experiment. Ampicillin is not required for elution, as can be seen by comparing lane 10 (without ampicillin) with lane 11 (10 mM ampicillin)—in fact the tag detaches itself of the ampicillin sepharose when incubated at temperatures between 0° C. and 10° C.

In FIG. 2, we show that some NaCl in the washing buffer is required to achieve high purity. However, moderate NaCl concentrations of 0.125 M are sufficient for very high purity of the transfected product, making this tag suitable for coprecipitation and pull down experiments for the discovery and study of physiological binding partners of the transfected protein of interest.

FIG. 3 shows that the presence of up to 30 mM ampicillin in the washing buffer does not affect purity or recovery of the fusion protein. This was somewhat unexpected, because I reasoned that unspecific ampicillin binding proteins should elute with ampicillin and therefore lead to enhanced sample purity. The result suggests that any residual impurities are not binding to the ligand but to the sepharose affinity medium. Importantly, ampicillin does not elute the tag.

In FIG. 4 it is shown that the catalytic domain (aa 37-297) of PBP5 is sufficient to facilitate the binding and elution properties of the protein. Like with the other experiments in the application the purification efficiency is very high. The Dac-tag (PBP5 aa 37-297) bound rapidly to ampicillin sepharose and the extract was depleted after 30 minutes of incubation. Although the dac-tag is robustly bound to ampicillin at ambient temperature, the bond can be broken rapidly simply by incubation for a few minutes on ice. Glycerol or Saccharose may be added to the elution buffer. This may accelerate elution at 4° C., but does not seem to make much difference when eluting on ice. Other experiments with bacterial cell extracts show that several penicillin binding proteins share these properties, implying that their penicillin binding domains may also be suitable for the use as affinity tags.

FIG. 5 demonstrates that the tag is not dependent on any particular buffer system. Commonly used buffers such as Tris, MOPS, HEPES and phosphate systems all work equally well. This tolerance to diverse buffer conditions make this system useful for a diverse range of applications.

FIG. 6 demonstrates that at ambient temperature the tag binds rapidly to ampicillin sepharose. Binding can be observed within minutes and the tag binds so fast that extracts may be loaded onto a packed column, where the ligand density is much higher. Therefore, with rapid binding, simple washing and fast elution this tag will be very useful for protein purification purposes.

FIG. 7 shows that the tag can be incubated over a period of time with ampicillin sepharose without loosing protein. Although long binding times may not be desirable for most applications, the observation that the tag stays robustly bound to the matrix for a period of time increases flexibility of use. Importantly binding at 4° C. is much lower than at ambient temperature. At 4° C. and in the presence of glycerol or sucrose the tag does not bind to ampicillin sepharose, a fact that can be exploited to facilitate elution.

FIG. 8 demonstrates that this purification system is not restricted to a human cell line, but can be used in different cell types. Here Dictostelium discordium was used, because it is a primitive eukaryote, hence quite unrelated to humans.

FIG. 9 shows that the tag can be used in another primitive eukaryote systems such as, for example, Saccharomyces cerevisiae (bakers yeast), further demonstrating its versatility.

FIG. 10 shows that the protein can be expressed in and purified from E. coli cells. The protein expresses at high levels when cells are incubated at 37° C. and can be purified to very high purity using a one step ampicillin chromatography procedure. In further experiments we find that when PBP5 aa 37-297 is preceded by a few aminoacids that give a sequence of low complexity, for example a poly His-tag, expression levels are very high, so that we obtained up to 40 mg protein per litre bacterial culture. This is probably due to much more efficient mRNA translation.

In summary, at ambient temperature (15° C.-37° C.), the penicillin binding domains of penicillin binding proteins bind fast and efficiently to beta lactam antibiotics. We observe that when the bond is cooled down it breaks down via a poorly understood mechanism. The mechanism most likely involves a change in the structure of the catalytic domain and may involve the hydrolysis of the ester bond that links the catalytic serine residue to the antibiotic. In some conditions hydroxyl containing reagents such as glycerol or sugar seem to accelerate the breakage of the bond, which may be exploited for efficient recovery of the product. At ambient temperature the penicillin binding proteins cannot be eluted with ampicillin, which strongly suggests that this is not an on/off reaction, but a solid covalent bond. However, in cold conditions, ampicillin is not required for elution, so that ampicillin binding proteins which bind non covalently, such as clathrin heavy chain proteins, elute in very small amounts by leakage. If the binding reaction is left for a short period of time, for example for 20 min and the elution is fast for example 3 times 5 minutes, then clathrin heavy chain is not detectable.

Whatever the molecular mechanism behind this, the control of the Dac-tag via temperature and the purification power of the Dac-tag are unique. Hence the dac-tag is useful for fast and efficient protein purification from any cell and in particular from eukaryotic cells.

### REFERENCES

Bush G. L., Tassin A. M., Fridén H., Meyer D. I. (1991) Secretion in yeast. Purification and in vitro translocation of chemical amounts of prepro-alpha-factor. J. Biol. Chem. 266:13811-13814.
Chen Y. T., Holcomb C, Moore H.-P. H. (1993) Expression and localization of two low molecular weight GTP-binding proteins, Rab8 and Rab 10, by epitope tag. Proc. Natl. Acad. Sci. USA. 90:6508-6512.
Clark A. G. Letoa M., Ting W. S. (1977) The purification by affinity chromatography of a glutathione S-transferase from larvae of Galleria mellonella. et al., 1977, Life Sci 20 141-147.
Crowe J., Dikeli H., Gentz R., Hochuli E., Stüber D., Henco K. (1994) 6×His-Ni-NTA chromatography as a superior technique in recombinant protein expression/purification. Methods Mol. Biol. 31:371-387.
Ellison M. J., Hochstrasser M. (1991) Epitope tagged Ubiquitin. J. Biol. Chem. 266:21160-21157.
Hochuli E, Döbeli A, Schacher A. (1987) New metal chelate adsorbent selective for proteins and peptides containing neighbouring histidine residues. J. Chromatography 411:177-184.
Honey S., Schneider B. L., Schieltz D. M., Yates J. R., Futcher B. (2001) A novel multiple affinity purification tag and its use in identification of proteins associated with a cyclin-CDK complex. Nucleic Acids Res. 29:E24.
Pratt J. M., Jackson M. E. Holland I. B. (1986) The C terminus of penicillin-binding protein 5 is essential for localisation to the E. coli inner membrane. EMBO J. 9: 2399-2405.
Puig O., Caspary F., Rigaut G., Rutz B., Bouveret E., Bragado-Nilsson E., Wilm M., Séraphin B (2001) The tandem affinity purification (TAP) method: a general procedure of protein complex purification. Methods. 24:218-229.
Rigaut G., Shevchenko A., Rutz B., Wilm M., Maim M., Séraphin B. (1999) A generic protein purification method for protein complex characterization and proteome exploration. Nature Biotechnol. 17:1030-1032.
Smith D. B., Johnson K. S. (1988) Single-step purification of polypeptides expressed in Escherichia coli as fusions with glutathione S-transferase. Gene 67:31-40.

1-29. (canceled)

30. A method of affinity purifying, isolating and/or extracting proteins, peptides and/or amino acids, said method comprising the steps of:
(a) contacting a sample comprising proteins, peptides and/or amino acids tagged or labelled with a beta-lactam (β-lactam) binding protein, with a compound capable of binding the β-lactam binding protein tag or label;
(b) removing any unbound protein or peptide; and
(c) separating the bound tagged or labelled protein, peptide or amino acid from the compound capable of binding the β-lactam binding protein tag or label.

31. The method of claim 30, wherein the β-lactam binding protein is a penicillin binding protein (PBP) or fragment, analogue, homologue, variant or derivative thereof.

32. The method of claim 30, wherein the β-lactam binding protein is selected from the group consisting of PBP1; PBP2a; PBP2b; PBP3; PBP4; PBP5 and PBP6.

33. The method of claim 30, wherein the β-lactam binding protein is selected from the group consisting of
(i) PBP5 encoded by the E. coli dacA gene;
(ii) residues 37-391 of PBP5 encoded by the E. coli dacA gene;
(iii) residues 37-297 of PBP5 encoded by the E. coli dacA gene; and
(iv) or a fragment, analogue, homologue, variant or derivative of (i)-(iii).

34. A protein, peptide or amino acid for affinity purification, tagged or labelled with a β-lactam binding protein.

35. The protein, peptide or amino acid of claim 34, tagged or labelled with PBP 5 or a β-lactam binding protein selected from the group consisting of:
(i) PBP5 encoded by the E. coli dacA gene;
(ii) residues 37-391 of PBP5 encoded by the E. coli dacA gene;
(iii) residues 37-297 of PBP5 encoded by the E. coli dacA gene; and
(iv) or a fragment, analogue, homologue, variant or derivative of (i)-(iii).

36. The method of claim 30, wherein the compound capable of binding the β-lactam binding protein tag or label is complexed to a support substrate.

37. The method of claim 30, wherein the compound capable of binding the β-lactam binding protein tag or label is complexed to a substrate is selected from the group consisting of agarose, sepharose, polyacrylamide, agarose/polyacrylamide co-polymers, dextran, cellulose, polypropylene, polycarbonate, nitocellulose, glass, paper and magnetic particles.

38. The method of claim 30, wherein the compound capable of binding the β-lactam binding protein tag or label is a β-lactam compound or a compound comprising a β-lactam ring.

39. The method of claim 30, wherein the compound capable of binding the β-lactam binding protein tag or label is, or comprises, one or more selected from the group consisting of:
(i) penicillin,
(ii) ampicillin
(iii) isopenicllin
(iv) cyclobutane
(v) cephalosporines
(vi) or derivatives, variants, analogues or homologues of any of (i)-(v).

40. The method of claim 30, wherein the bound tagged or labelled proteins are separated from the compound capable of binding the β-lactam binding protein tag or label by contact with a buffer having a temperature of between approximately 1° C. and 10° C.

41. The method of claim 40, wherein the buffer comprises one or more selected from the group consisting of:
(i) an alcohol;
(ii) glycerol;
(iii) a sugar;
(iv) sucrose;
(v) glucose;
(vi) fructose;
(vii) saccharose;
(viii) ionic agents; and
(ix) detergents.

42. A vector comprising a nucleic acid sequence encoding a β-lactam binding protein or fragment, variant, analogue or derivative thereof, for the expression of proteins, peptides or amino acids tagged or labelled with a β-lactam binding protein.

43. A cell transfected or transformed with the vector of claim 42.

44. A method of producing or generating a protein, peptide or amino acid tagged or labelled with a β-lactam binding protein, said method comprising the steps of introducing into a cell a nucleic acid sequence encoding a protein, peptide or amino acid fused to a β-lactam binding peptide and maintaining the cell under conditions suitable to permit or induce expression of the fused protein or peptide.

45. An in vitro method of detecting a compound tagged or labelled with a β-lactam binding protein, said method comprising the steps of
(i) contacting a compound with a compound capable of binding a β-lactam binding protein under conditions which permit binding between compounds tagged or labelled with a β-lactam binding protein and compounds capable of binding thereto;
(ii) removing unbound compound capable of binding a β-lactam binding protein; and
(iii) detecting bound compound capable of binding a β-lactam binding protein.


SEQUENCE LISTING (3 sequences)

SEQ ID NO: 1 - protein, 403 aa, Escherichia coli
MNTIFSARIMKRLALTTALCTAFISAAHADDLNIKTMIPGVPQIDAESYILIDYNSGKVLAEQNADVRRDPASLTKMMTSYVIGQAMKAGKFKETDLVTIGNDAWATGNPVFKGSSLMFLKPGMQVPVSQLIRGINLQSGNDACVAMADFAAGSQDAFVGLMNSYVNALGLKNTHFQTVHGLDADGQYSSARDMALIGQALIRDVPNEYSIYKEKEFTFNGIRQLNRNGLLWDNSLNVDGIKTGHTDKAGYNLVASATEGQMRLISAVMGGRTFKGREAESKKLLTWGFRFFETVNPLKVGKEFASEPVWFGDSDRASLGVDKDVYLTIPRGRMKDLKASYVLNSSELHAPLQKNQVVGTINFQLDGKTIEQRPLVVLQEIPEGNFFGKIIDYIKLMFHHWFG

SEQ ID NO: 2 - DNA, 32 bp, Artificial
  Primer
atcgctagccaccatgatcccgggtgtaccgc

SEQ ID NO: 3 - DNA, 59 bp, Artificial
  Primer
gtaagcttgggcccctggaacagaacttccagatcaatgattttgccgaagaagttacc